(19) United States
(12) Patent Application Publication
(10) Pub. No.: US 2026/0232877 A1
(43) Pub. Date: Aug. 13, 2026

(54) Functionalized Metallic Implants with Covalently Bound Polymer Coatings For Therapeutic Agent Association

(71) Applicant: Onkos Surgical, Inc., Parsippany, NJ (US)

(72) Inventors: Patrick Treacy, Parsippany, NJ (US); Liam Sweeney, Parsippany, NJ (US); Arley Perez, III, Parsippany, NJ (US); Gene Kulesha, Parsippany, NJ (US); Ryan J. Hue, Parsippany, NJ (US)

(73) Assignee: Onkos Surgical, Inc., Parsippany, NJ (US)

(21) Appl. No.: 19/538,411

(22) Filed: Feb. 12, 2026

Related U.S. Application Data
(60) Provisional application No. 63/758,113, filed on Feb. 13, 2025.

(51) Int. Cl. A61L 31/16; A61K 9/00; A61L 27/54; A61L 29/16

(52) U.S. Cl. CPC A61L 31/16; A61K 9/0024; A61L 27/54; A61L 29/16

(57) ABSTRACT

The present invention relates to a functionalized metallic implant comprising a metallic substrate having a covalently bound organic acid layer and a polymeric coating covalently attached thereto. The polymeric coating includes cationic, anionic, zwitterionic, and/or hydrophobic functional groups configured to associate therapeutic agents via electrostatic and/or hydrophobic interactions. Therapeutic agent release or availability is controlled as a function of surface charge, hydrophobicity, or both, enabling localized and sustained delivery in response to local biological conditions. In certain embodiments, the coating provides antibacterial activity through contact-based membrane disruption or inhibition of bacterial adhesion. In further embodiments, zwitterionic coatings exhibit pH-responsive charge behavior enabling selective loading and release of therapeutic agents. The invention further relates to methods of forming and using the implant for localized delivery of antimicrobial agents, gene therapies, and growth factors.

### CROSS-REFERENCE TO RELATED APPLICATIONS

This application claims the benefit of US Provisional Application No. 63/753,113, filed on February 13, 2025, which application is hereby incorporated herein by reference.

### TECHNICAL FIELD

The present invention relates to metallic implants with functionalized coatings that provide antibacterial properties, controlled therapeutic agent delivery, and enhanced biocompatibility.

### BACKGROUND

Metallic implants are widely used in orthopedic, dental, spinal, trauma, and vascular applications to restore function and support biological structures. However, these implants are prone to bacterial colonization and biofilm formation, leading to periprosthetic joint infections (PJIs), implant failures, and surgical complications. Traditional antimicrobial coatings rely on eluting agents that lose effectiveness over time, may contribute to antibiotic resistance, and can result in systemic exposure. Moreover, current approaches to local drug delivery from implants are constrained by binding instability, uncontrolled release kinetics, and surface modifications that degrade or elute over time, thereby limiting long-term therapeutic efficacy.

Accordingly, there remains a need for an implant coating that provides a permanent, non-eluting antimicrobial function while simultaneously enabling site-specific, controlled release of therapeutic agents.

### SUMMARY

The present invention addresses these challenges by covalently tethering a polymeric coating to a metallic implant surface via an organic acid layer. The coating includes cationic, anionic, zwitterionic, or hydrophobic functional groups, allowing for electrostatic and hydrophobic binding of therapeutic agents.

In certain embodiments, the polymeric coating is formed by surface-initiated polymerization from the organic acid layer. This system may provide antibacterial activity by disrupting bacterial membranes upon contact, while also enabling controlled therapeutic agent delivery in response to local biological conditions such as pH, ionic strength, or lipid interactions. The invention offers a novel, stable, and multifunctional approach to infection prevention, drug delivery, and enhanced implant performance in a wide range of medical applications.

The present invention relates to such a functionalized metallic implant incorporating a covalently bound polymeric coating that provides antibacterial properties, controlled drug delivery, and enhanced implant performance. The implant comprises a metallic substrate, such as titanium, cobalt-chrome, stainless steel, or magnesium-based bioresorbable metal, onto which an organic acid layer, preferably phosphonic acid, is covalently bound. A polymeric coating is then covalently attached to the organic acid layer, wherein the polymer comprises cationic, anionic, zwitterionic, or hydrophobic functional groups, enabling electrostatic and hydrophobic binding of therapeutic agents. The organic acid layer may comprise phosphonic acid, carboxylic acid, or siloxane, with phosphonic acids being preferred due to their superior hydrolytic stability. The organic acid or siloxane may have a hydrocarbon chain length ranging from C2 to C30 and may terminate in functional groups including hydroxyl, halogenated alkyl, amine, pyridine, methacrylate, or acrylate groups.

The invention further provides a method of forming the functionalized metallic implant, comprising covalently binding the organic acid layer to the metallic substrate, polymerizing the functionalized coating via surface-initiated polymerization, and associating therapeutic agents with the polymeric coating. The polymerization may be performed using Atom Transfer Radical Polymerization (ATRP), or Reversible Addition-Fragmentation Chain Transfer (RAFT) polymerization, or related controlled radical polymerization techniques. The monomers utilized may include methacrylate, methacryloyl, methacrylamide, acrylate, or acryloyl functional groups capable of free-radical polymerization. The polymeric coating enables the electrostatic binding of oppositely charged therapeutic agents and the hydrophobic retention of lipophilic or nonpolar drugs, allowing for controlled release based on surface charge, hydrophobicity, and surrounding biological conditions.

Additionally, the invention discloses a method of using the metallic implant, wherein the implant is surgically implanted at a treatment site. Bacteria present at, or migrating to, the treatment site may be contacted by the polymeric coating, wherein cationic functional groups disrupt bacterial cell membranes or inhibit bacterial adhesion, reducing biofilm formation. Therapeutic agents associated with the polymeric coating may be released into surrounding tissue in response to environmental factors such as pH, ionic strength, or lipid interactions. The invention is applicable to orthopedic, dental, spinal, trauma, tumor, and vascular implants, providing infection prevention, site-specific drug delivery, and improved implant longevity.

Unless otherwise expressly indicated, any feature, element, component, or step described herein with respect to one embodiment may be combined with any feature, element, component, or step described with respect to one or more other embodiments, provided such combination is technically compatible. In particular, embodiments relating to the composition of the organic acid layer, the nature of the polymeric coating (including cationic, anionic, zwitterionic, hydrophobic, or mixed functional groups), the type of therapeutic agent, the method of therapeutic agent association, the mechanism of release or availability, and the method of use may be combined in any technically compatible manner to form further embodiments. All such combinations are intended to fall within the scope of the present disclosure.

### BRIEF DESCRIPTION OF THE DRAWINGS

The accompanying figures, which are incorporated in and constitute a part of the specification, illustrate preferred embodiments of the invention and, together with the description, serve to explain the objects, advantages, and principles of the invention. Embodiments of the invention are in no way limited by the following figures:

FIG. 1A illustrates an example of a positively charged antibacterial coating on a metallic implant, featuring quaternary ammonium functional groups such as Methacryloyloxydodecyl Pyridinium Bromide (MDPB) that disrupt bacterial membranes and reduce colonization;

FIG. 1B illustrates an example of a negatively charged coating incorporating methacrylic acid functional groups on a metallic implant, providing hydrophilic properties and electrostatic repulsion to inhibit bacterial adhesion and biofilm formation.

FIG. 1C illustrates an example of a zwitterionic polymer coating with sulfobetaine methacrylate groups that form a hydration layer, preventing protein adsorption and bacterial adhesion while incorporating hydrophobic regions for surface stability and maintaining long-term antimicrobial properties.

FIG. 2 illustrates a metallic implant having a positively charged coating, as described in FIG. 1A, before treatment and after exposure to a povidone iodine solution, showing association of iodine with the coated surface following treatment.

FIG. 3 is a graph illustrating X-ray Photoelectron Spectroscopy (XPS) spectra of MDPB-coated cobalt chrome surfaces, comparing a coated sample exposed to a povidone iodine solution with a coated control sample not exposed to povidone iodine. In practice, the coated surface may exhibit a yellow to yellow-brown coloration due to iodine uptake; however, the figure schematically indicates iodine binding without reliance on color.

FIG. 4A is a schematic illustration of controlled gene therapy delivery from a metallic implant surface having a positively charged polymeric coating, wherein negatively charged nucleic acid material electrostatically binds to the coating following exposure of the implant to a gene therapy solution.

FIG. 4B is a schematic illustration of controlled gene therapy delivery from a metallic implant surface having a negatively charged polymeric coating, wherein positively charged nucleic acid material electrostatically binds to the coating following exposure of the implant to a solution containing positively charged gene therapy complexes.

FIG. 5 is a schematic illustration of electrostatic binding of a positively charged bioactive agent to a negatively charged polymeric coating disposed on a porous metallic implant, for example bone morphogenetic protein-2 (BMP-2).

FIG. 6A is a schematic illustration of a zwitterionic polymeric coating disposed on a metallic implant surface, illustrating electrostatic association of a positively charged antiseptic to the coating under basic or relatively alkaline pH conditions.

FIG. 6B is a schematic illustration of the zwitterionic polymeric coating of FIG. 6A following implantation in a surgical site, illustrating antiseptic release or availability and contact-based antimicrobial activity or inhibition of bacterial adhesion in response to local environmental changes, such as pH variation associated with bacterial presence.

### DETAILED DESCRIPTION OF ILLUSTRATIVE EMBODIMENTS

The present disclosure relates to metallic implant surfaces functionalized with covalently bound organic and polymeric coatings that provide antimicrobial activity and controlled therapeutic agent delivery. In various embodiments, the surface properties of the implant are tailored through the polymerization of cationic, anionic, zwitterionic, and/or hydrophobic monomers from a covalently attached linking layer, enabling selective binding and controlled release of therapeutic agents based on electrostatic and hydrophobic interactions.

The following figures illustrate representative embodiments of the invention and are described in detail below. These embodiments are provided for illustrative purposes and are not intended to limit the invention to the specific structures, materials, or methods shown.

### Base Coating Structure and Formation

The functionalized coating of the present invention comprises a multi-layer architecture designed to provide stable, covalent attachment to metallic implant surfaces while enabling controlled therapeutic agent binding and release.

### Layer 1: Covalently Bound Linking Layer

The first layer of the coating comprises a covalent attachment layer to a metal oxide surface of the implant. This layer may be formed from any organic acid, including phosphonic acid or carboxylic acid, or from a siloxane. Phosphonic acids are preferred due to their superior hydrolytic stability compared to other organic acids or siloxanes.

A self-assembled monolayer (SAM) is formed through controlled dip coating of the metallic substrate from an organic acid or siloxane solution. During this process, the hydrophilic head of the organic acid or siloxane aligns with the hydrophilic metal oxide surface of the implant. The metallic substrates are then heated under vacuum at temperatures ranging from approximately 100°C to 200°C to drive conversion to covalent metal-oxygen-phosphorus bonds (in the case of phosphonic acids) or analogous covalent bonds for other organic acids or siloxanes.

In one embodiment, the SAM comprises a phosphonic acid with a terminal alcohol, such as 11-hydroxyundecylphosphonic acid (PUL). However, a wide variety of phosphonic acids may be utilized with various functional groups allowing further modification. Suitable terminal functional groups include halogenated alkyl groups (Br, Cl, I), amine groups, pyridine groups, methacrylate groups, or acrylate groups. The length of the hydrocarbon chain for the organic acid or siloxane may range from C2 to C30.

Following formation of the SAM, the metal substrates are sonicated in reagent alcohol to remove any physiosorbed (non-covalently bound) organic acids or siloxanes, ensuring that only covalently bound molecules remain on the surface.

### Layer 2: Functionalization of SAM Layer

The covalent attachment layer of the coating may be further functionalized to enable controlled polymerization of charged or hydrophobic monomers from the surface. In one embodiment, the terminal alcohol of PUL is converted with an atom transfer radical polymerization (ATRP) initiator, such as alpha-bromoisobutyryl bromide (BiBB). This reaction is performed in a halogenated solvent, such as dichloromethane, with an amine catalyst, such as triethylamine, to promote grafting of the initiator to the terminal alcohol.

Following the initiator grafting reaction, the parts are rinsed in reagent alcohol multiple times (e.g., three times), then sonicated in reagent alcohol (e.g., twice) to remove any adsorbed initiator, catalyst, or solvent. This ATRP initiator serves as a surface initiation site for surface-initiated ATRP, enabling the controlled polymerization of any monomer with a functional group capable of free-radical polymerization. Example functional groups for such monomers include methacrylate, methacryloyl, methacrylamide, acrylate, and acryloyl groups. In certain embodiments, this functionalization step defines the density and location of polymer growth sites on the implant surface.

Alternative functionalization approaches may be employed depending on the desired polymerization technique. For example, instead of an ATRP initiator, a RAFT agent may be installed at the terminal alcohol of the SAM for RAFT polymerization.

### Layer 3: Charged or Hydrophobic Polymeric Layer

Once the polymerization initiator or agent is grafted to the opposing end of the SAM, polymerization of charged or hydrophobic monomers may be performed to alter the surface charge or hydrophobicity of the implant. These monomers may be polymerized using various polymerization techniques, including ATRP, reversible addition–fragmentation chain transfer (RAFT) polymerization, free-radical polymerization, and anionic polymerization.

In one embodiment, the coating comprises a positively charged quaternary ammonium methacrylate monomer, such as 12-methacryloyloxydodecyl pyridinium bromide (MDPB). This monomer may be polymerized (e.g., via ATRP) from the surface to form a brush coating. The resulting polymer provides permanent, non-eluting antimicrobial protection by disrupting bacterial cell membranes upon contact.

The monomer chosen for polymerization may alternatively be negatively charged, such as methacrylic acid, creating a highly negatively charged surface when polymerized from the SAM surface. The monomer may also be zwitterionic, such as sulfobetaine methacrylate or carboxybetaine methacrylate, allowing for a mixed charge surface where the net charge changes based on physiological conditions such as pH and temperature.

Multiple monomers may be used together in the polymerization to create a tuned, mixed charge surface as desired based on the therapeutic agent of interest. This enables precise control over the electrostatic and hydrophobic properties of the implant surface.

The present invention relates to the tethering of therapeutic agents to covalently bound charged surfaces of metallic implants, providing antibacterial properties, enhanced biocompatibility, and controlled therapeutic agent delivery. The invention utilizes a covalently bound organic acid layer, upon which a charged or hydrophobic polymer coating is attached through the polymerization of cationic, anionic, zwitterionic, and/or hydrophobic monomers.

This functionalized surface enables the stable tethering of therapeutic agents via electrostatic or hydrophobic interactions, allowing for controlled release in response to the surrounding biological environment, ensuring localized and sustained therapeutic efficacy.

The present invention begins with a covalently bound organic acid to a metallic implant surface, to which a charged or hydrophobic coating is covalently bound through polymerization of a cationic, anionic, zwitterionic, and/or hydrophobic monomer. A therapeutic agent of a certain charge or hydrophobic property is applied to the coating where the substance is electrostatically attracted to the charged/hydrophobic surface.

The release of the therapeutic agent from the implant is regulated by the surrounding medium, allowing for controlled and site-specific drug delivery. Factors such as pH, ionic strength, temperature, and lipid interactions influence the electrostatic or hydrophobic binding dynamics of the therapeutic agent to the polymeric coating. In certain embodiments, release is controlled as a function of the surface charge and/or hydrophobicity of the polymeric coating.

In certain embodiments, the functionalized implant is formed by preparing the metallic substrate, covalently attaching an organic acid or siloxane layer to create a stable functionalized surface, and forming the polymeric coating by surface-initiated polymerization, including ATRP, RAFT polymerization, or other free-radical polymerization techniques. Therapeutic agents may then be associated with the polymeric coating through electrostatic and/or hydrophobic interactions to provide localized and controlled delivery at a treatment site.

### Overview Of Functionalized Implant Coatings

### Surface Charge Configurations of Polymeric Implant Coatings

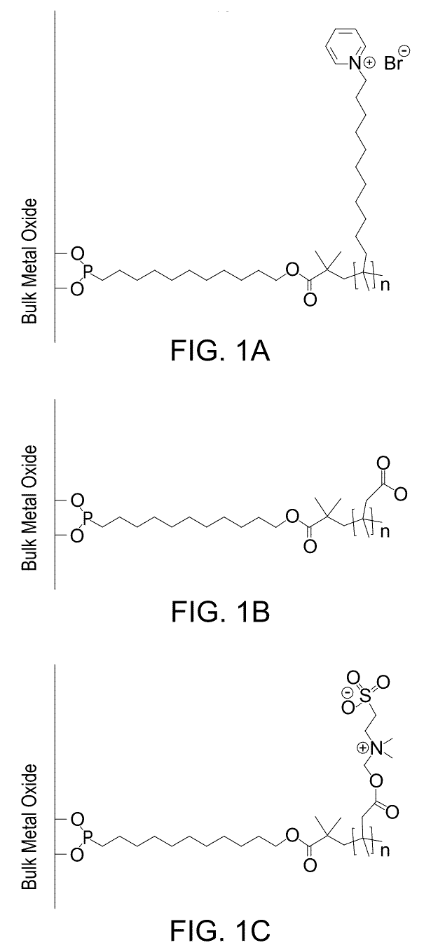

FIG. 1A illustrates a positively charged antibacterial coating applied to a metallic implant. This coating comprises quaternary ammonium functional groups, including Methacryloyloxydodecyl Pyridinium Bromide (MDPB), which impart a permanent positive charge to the surface. The positive charge facilitates bacterial membrane disruption, reducing bacterial adhesion and colonization. The polymerization of this coating is achieved using Atom Transfer Radical Polymerization (ATRP); however, other free radical polymerization techniques, such as reversible addition–fragmentation chain transfer (RAFT) polymerization or conventional radical polymerization, may be employed to achieve similar functional properties. The coating may also incorporate hydrophobic regions to enhance surface stability and antimicrobial effectiveness.

FIG. 1B illustrates a negatively charged coating applied to a metallic implant, incorporating methacrylic acid functional groups that provide hydrophilicity and electrostatic repulsion. The negatively charged groups (-COO⁻) create a surface that resists bacterial adhesion and biofilm formation by repelling bacteria with negatively charged membranes. This coating can be synthesized using ATRP or other free radical polymerization methods. Additionally, hydrophobic regions within the polymer structure may enhance durability and resistance to protein fouling, improving the long-term performance of the implant.

FIG. 1C illustrates a zwitterionic polymer coating applied to a metallic implant, incorporating sulfobetaine methacrylate functional groups. This zwitterionic structure, containing both positive (N⁺) and negative (SO₃⁻) charges, enables the formation of a hydration layer that prevents non-specific protein adsorption, bacterial adhesion, and biofilm formation. The hydration layer mimics the natural antifouling properties of cell membranes, enhancing biocompatibility and long-term antimicrobial effectiveness. The coating is synthesized via ATRP or other free radical polymerization techniques and includes hydrophobic regions, which contribute to surface stability and durability. In one embodiment, the zwitterionic monomer comprises carboxybetaine methacrylate, wherein the quaternary ammonium group dominates at acidic pH and the carboxylic acid group dominates at basic pH. This pH-responsive behavior enables controlled release of therapeutic agents in response to infection-induced pH changes in the surgical site.

### Therapeutic Agent Binding and Controlled Release Mechanisms

The charged polymeric coating enables electrostatic binding of therapeutic agents having an opposite charge to the coating surface. For example, a highly cationic surface created using MDPB polymerization can electrostatically bind negatively charged therapeutic agents. This electrostatic binding mechanism was demonstrated when MDPB-coated implants were exposed to povidone iodine solution containing polyvinylpyrrolidone (PVP) complexed with negatively charged iodide ions. The negative charge potential of the PVP and iodide ions was sequestered to the positively charged surface, demonstrating electrostatic binding as evidenced by the yellow to yellow-brown coloration of the metallic surface and XPS analysis confirming iodide ions on the surface.

The cationic surface provides improved binding of povidone iodine while also controlling the release from the implant surface. The cationic surface electrostatically retains the negatively charged iodide ions, causing them to be released more slowly under physiological conditions compared to an implant with a net neutral or net negative charge. This controlled release may extend over a period of approximately 1 to 4 weeks.

### Electrostatic Binding of Therapeutic Agents to Charged Coatings

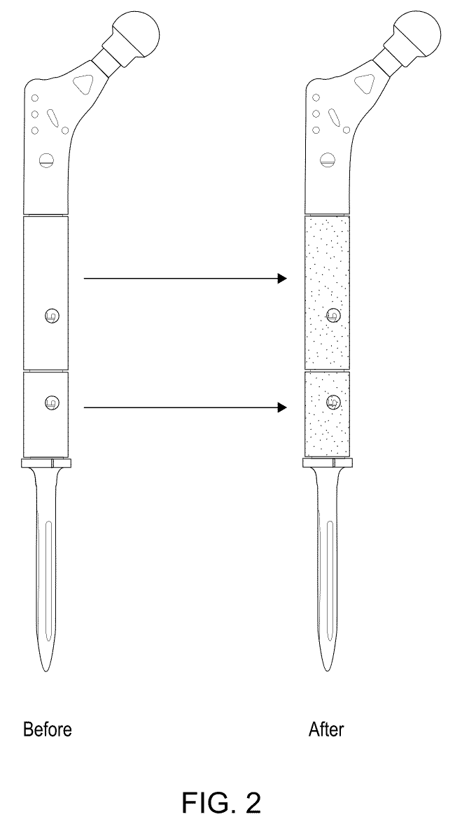

FIG. 2 illustrates a metallic implant component having a positively charged polymeric coating before and after exposure to a povidone iodine solution. The implant shown on the left (“Before”) includes the positively charged coating, as described in FIG. 1A, prior to treatment with the iodine-containing solution.

As shown on the right (“After”), following exposure of the coated implant to a povidone iodine solution (for example, by immersion in a solution having a concentration of approximately 0.1% to 10% and optional subsequent rinsing with deionized (DI) water), the coated regions of the implant exhibit a visible change in appearance schematically indicating association of iodine species with the positively charged coating. This association is consistent with electrostatic binding of negatively charged iodide ions and interaction of elemental iodine with hydrophobic regions of the polymeric coating.

In practice, the coated region of the implant may exhibit a yellow to yellow-brown coloration due to iodine uptake. The iodine association is consistent with electrostatic binding of negatively charged iodide ions (I⁻) to the positively charged surface and association of elemental iodine (I₂) with hydrophobic hydrocarbon chain regions of the coating. In contrast, an uncoated control implant lacking the positively charged polymer layer shows limited to no iodine association under similar exposure conditions, demonstrating that electrostatic and hydrophobic interactions of the coating facilitate enhanced iodine retention.

The described coating approach enables iodine retention that may provide antibacterial properties, thereby aiding in infection prevention for implanted medical devices. The coating compositions and iodine association mechanisms described herein may be applied to a variety of metallic implants used in orthopedic, trauma, tumor, revision, cardiovascular, neurological, dental, and craniofacial procedures, as well as other implantable medical devices, while maintaining biocompatibility.

### Analytical Confirmation of Therapeutic Agent Association

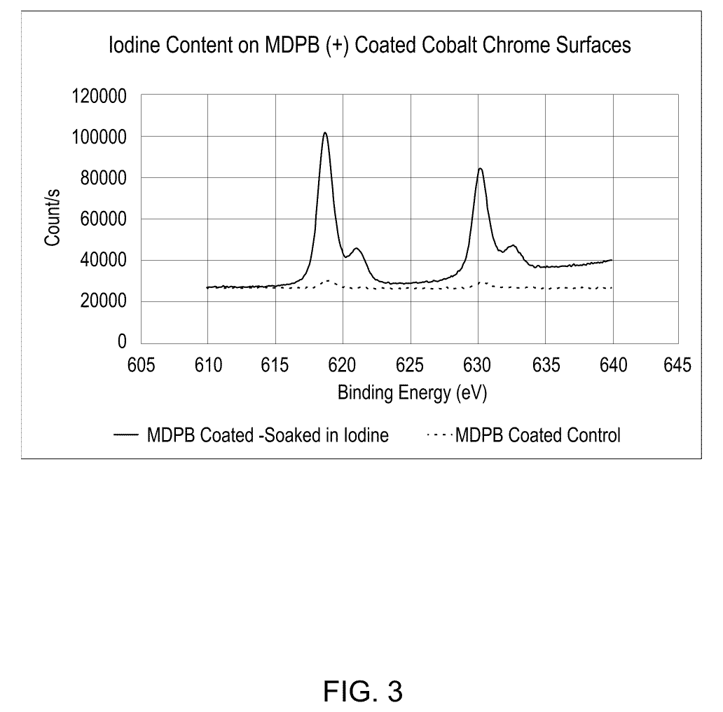

FIG. 3 illustrates the iodine content on MDPB-coated cobalt chrome surfaces, analyzed using X-ray Photoelectron Spectroscopy (XPS) to confirm the presence of iodine associated with the positively charged surface. The coated sample was prepared as described with respect to FIG. 1A, featuring a quaternary ammonium-functionalized polymer layer that provides a permanent positive charge to the surface.

In one example, to assess iodine retention, a coated implant was exposed to a povidone iodine solution (e.g., a 10% solution) and optionally rinsed with deionized (DI) water prior to analysis. The XPS spectra show a strong iodine signal for the treated sample relative to a control sample not exposed to povidone iodine, indicating association of iodine species with the positively charged coating. The observed iodine association is consistent with electrostatic interaction of iodide ions (I⁻) with the positively charged surface and association of elemental iodine (I₂) with hydrophobic hydrocarbon chain regions of the coating.

These results demonstrate that the positively charged surface is capable of retaining iodine, which may contribute to antimicrobial properties by enabling sustained iodine availability. The described coatings and iodine association mechanisms may be applied to a variety of metallic implants, including those used in orthopedic, trauma, tumor, revision, cardiovascular, neurological, dental, and craniofacial procedures, while maintaining biocompatibility.

### Controlled Release and Clinical Use

The release or availability of therapeutic agents from the implant may extend over various time periods depending on the therapeutic agent and coating configuration. For example, for povidone iodine and gene therapies, controlled release may be provided for approximately 1 to 4 weeks, while for growth factors such as BMP-2, therapeutic availability may be maintained for longer periods, such as approximately 2 months. The concentration or mass of therapeutic agents associated with the implant surface may range from approximately microgram to milligram quantities, depending on the specific therapeutic agent and application.

In clinical use, the implant may be surgically placed at a treatment site, such as a bone, joint, or vascular region. The cationic functional groups of the polymer coating may interact with bacterial cell membranes upon contact, which can inhibit bacterial colonization and biofilm formation. Meanwhile, therapeutic agents associated with the coating may be released or become available in response to local biological stimuli, providing localized infection prevention, anti-cancer treatment, or regenerative support as needed. Unlike traditional antimicrobial coatings that rely on elution-based drug release, the polymeric coating remains permanently attached to the implant, which may enable long-term antimicrobial function and therapeutic delivery.

### Drug Delivery Applications

### I. Antimicrobial Agent Delivery

In certain embodiments, the functionalized implant surfaces described herein are configured to bind and deliver antimicrobial agents in a localized and controlled manner. By tailoring the surface charge and hydrophobicity of the polymeric coating, antimicrobial agents may be electrostatically and/or hydrophobically associated with the implant surface, enabling controlled availability or sustained release at a surgical site while reducing systemic exposure. This approach may provide antimicrobial protection of the implant and surrounding tissue and may be used with a wide range of antiseptics, antibiotics, and antimicrobial compounds.

### A. Povidone Iodine Delivery

Positively charged surfaces may be utilized to deliver povidone iodine in a controlled manner. The povidone iodine may be electrostatically associated with the cationic surface and made available over periods on the order of approximately 1 to 4 weeks, rather than through immediate systemic delivery which would occur when applied to a neutral or negatively charged implant or irrigated surgical site. This formulation may provide sustained antimicrobial activity of the implant while also delivering povidone iodine to the surgical site.

### B. Alternative Antimicrobial Agent Delivery

In addition to povidone iodine, the functionalized implant surfaces described herein may be utilized to bind and deliver other antimicrobial agents having a charge or hydrophobic character complementary to the polymeric coating. In certain embodiments, positively charged polymeric coatings may electrostatically bind or associate with negatively charged antimicrobial agents, while negatively charged coatings may bind or associate with positively charged antimicrobial agents. In further embodiments, hydrophobic regions of the polymeric coating may associate with nonpolar or amphiphilic antimicrobial compounds via hydrophobic interactions.

Suitable antimicrobial agents include, but are not limited to, chlorhexidine, polyhexamethylene biguanide (PHMB), silver-containing compounds, antibiotic molecules, antiseptics, and combinations thereof. The antimicrobial agents may be applied to the implant surface through soaking, spraying, or other application techniques prior to implantation. Once implanted, the antimicrobial agents may become available over time or released in a controlled manner, providing localized infection prevention while minimizing systemic exposure.

### II. Gene Therapy Delivery

Cationic polymers have been utilized for delivery of gene therapies due to their ability to complex negatively charged nucleic acids of DNA and RNA. Once complexed, cationic polymers may facilitate delivery of DNA or RNA to target cells. A highly cationic polymeric brush surface provides an implant surface that may act as a local reservoir for association and delivery of gene therapies.

### A. Direct Gene Therapy Delivery from Cationic Surfaces

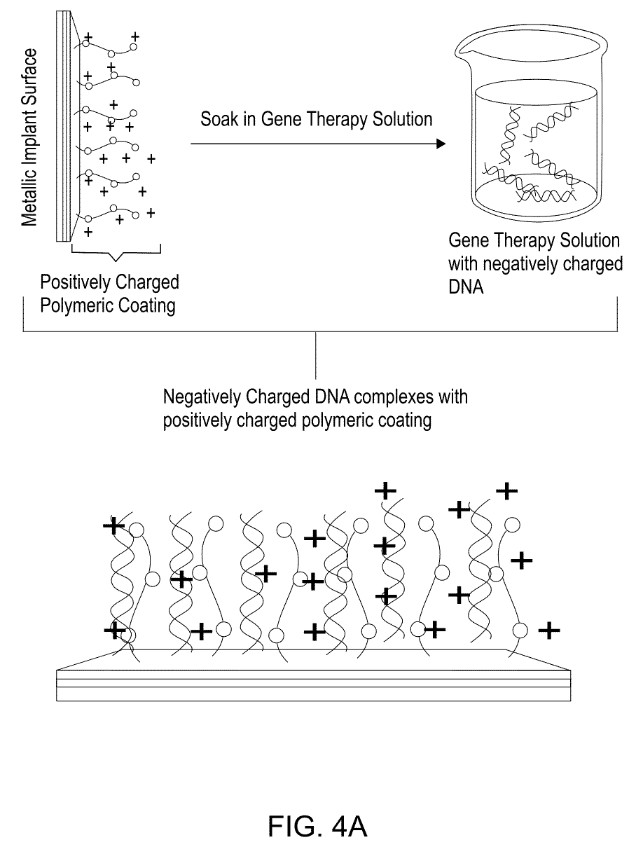

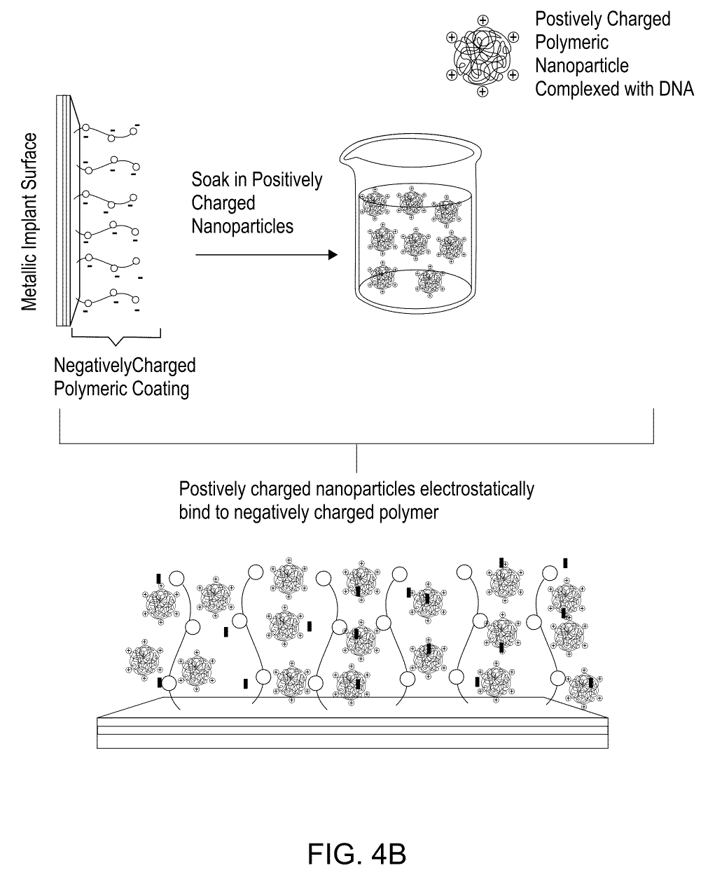

FIG. 4A illustrates one embodiment of direct gene therapy delivery from a metallic implant having a positively charged polymeric coating. As shown, the implant surface is functionalized with a cationic polymer coating and is exposed (e.g. via immersion or spraying) in a gene therapy solution containing negatively charged nucleic acids, such as DNA or RNA. During soaking or spraying, the negatively charged nucleic acids electrostatically associate with and optionally complex with the positively charged polymeric coating, forming surface-bound gene therapy complexes that remain associated with the implant following removal from the solution.

This application is of particular interest for anti-cancer therapeutics. For example, following resection of a bone tumor in the tibia or femur, a metallic stem may be placed intramedullary to fix a joint replacement. The stem or joint replacement may comprise the cationic brush coating described herein and may be soaked or sprayed with an anticancer gene therapy prior to implantation. The positively charged coating associates with the gene therapy and enables localized delivery of the gene therapy to remaining cancer cells not eradicated through resection or systemic chemotherapy. Therapeutic availability of the gene therapy may be maintained for periods on the order of up to approximately 4 weeks, and the concentration or mass of nucleic acid bound to the implant surface may range from approximately microgram to milligram quantities.

### B. Direct Gene Therapy Delivery from Anionic Surfaces

FIG. 4B illustrates an alternative embodiment of gene therapy delivery in which a negatively charged polymeric coating is provided on a metallic implant surface. In this embodiment, the implant surface is functionalized with an anionic polymer coating and is exposed in a solution containing gene therapies complexed with positively charged polymers, commonly in the form of nanoparticles. As shown, the positively charged nanoparticles electrostatically bind or associate with the negatively charged polymeric coating during soaking, spraying, or other application methods, forming surface-associated nanoparticle complexes on the implant.

In vivo, the implant may provide localized or controlled delivery of the nanoparticles containing the gene therapy to surrounding tissue, including remaining cancer cells not eradicated through surgical resection or systemic chemotherapy. The electrostatic interaction between the negatively charged coating and the positively charged nanoparticles may influence availability or release of the gene therapy over time, which may extend for periods of up to approximately 4 weeks. The concentration or mass of nanoparticles bound to the implant surface may range from approximately 1 microgram to milligram quantities.

### III. Growth Factor Delivery

Metallic implants typically provide structural support and may facilitate cell attachment but do not inherently promote tissue regeneration. This is particularly relevant for implants intended to support bone or soft tissue ingrowth. The charge state of certain growth factors can influence their ability to associate with and be released from implant surfaces in vivo. By controlling the net surface charge of the implant, growth factors may be electrostatically associated with the implant surface and delivered in a localized and controlled manner, with therapeutic availability or release profiles that may extend for periods on the order of up to approximately 2 months.

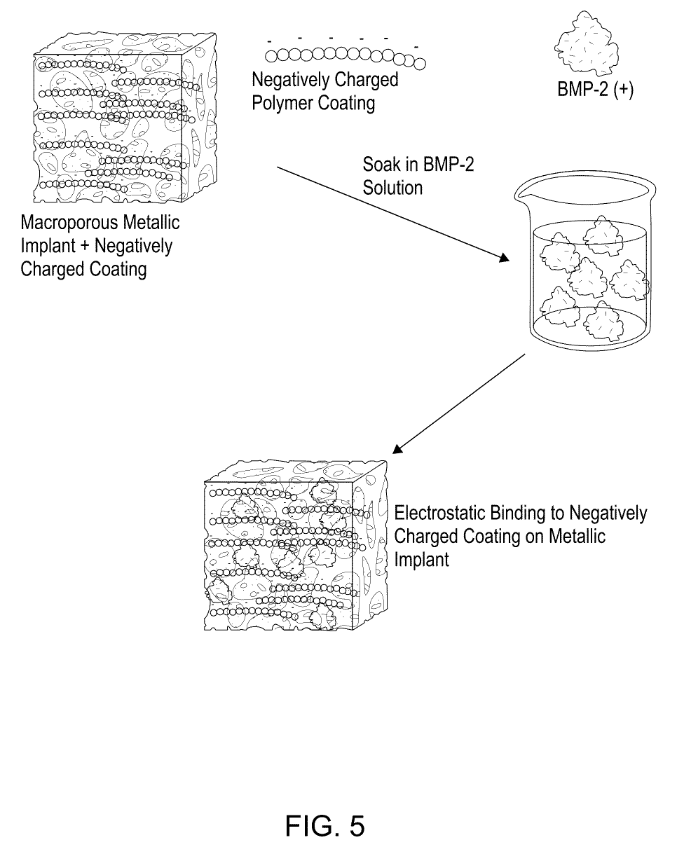

FIG. 5 illustrates one embodiment of growth factor delivery using a negatively charged polymeric coating applied to a metallic implant, including a porous or macroporous implant structure. In this embodiment, the implant surface is functionalized with an anionic polymer coating, for example by polymerizing a negatively charged monomer such as methacrylic acid from a self-assembled monolayer (SAM). The coated implant is exposed (e.g., by immersion) to a solution containing a positively charged growth factor, such as bone morphogenetic protein-2 (BMP-2), which may exhibit a net positive charge under physiological conditions. During exposure, the growth factor may electrostatically associate with the negatively charged polymeric coating, including association along surfaces within pores of the implant.

The electrostatic interaction between the negatively charged coating and BMP-2 may enable retention of the growth factor on the implant surface and within the porous structure, thereby allowing for controlled availability or release over time following implantation. This approach is particularly advantageous for porous implants designed to promote bone ingrowth, as the localized delivery of BMP-2 may support bone regeneration and integration of the implant. While BMP-2 is shown as an illustrative example, this approach may be applied to other positively charged growth factors or regenerative biomolecules as desired.

### IV. Zwitterionic Coating Controlled Release

Zwitterionic polymers comprise both positively charged and negatively charged functional groups within the same polymer structure. The dominant surface charge of such polymers can change in response to environmental conditions, most notably pH (and in some embodiments temperature). This dynamic charge behavior may enable responsive therapeutic binding and release, thereby supporting infection-responsive antimicrobial protection for permanent metallic implants.

### A. Zwitterionic Polymer Charge Switching

In certain embodiments, a metallic implant is provided with a zwitterionic polymer coating formed from a zwitterionic monomer selected such that the relative dominance of cationic and anionic functional groups varies with pH. One example of a suitable zwitterionic monomer is carboxybetaine methacrylate. At basic or physiological pH, the carboxylate group may predominate, imparting a net negative surface character, whereas at acidic pH the quaternary ammonium group may become dominant, imparting a net positive surface character.

The zwitterionic monomer may be polymerized from a covalently bound organic acid layer, such as a phosphonic acid SAM, to form a polymer brush coating on the metallic implant surface.

### B. Antiseptic Loading Under Non-Infected Conditions

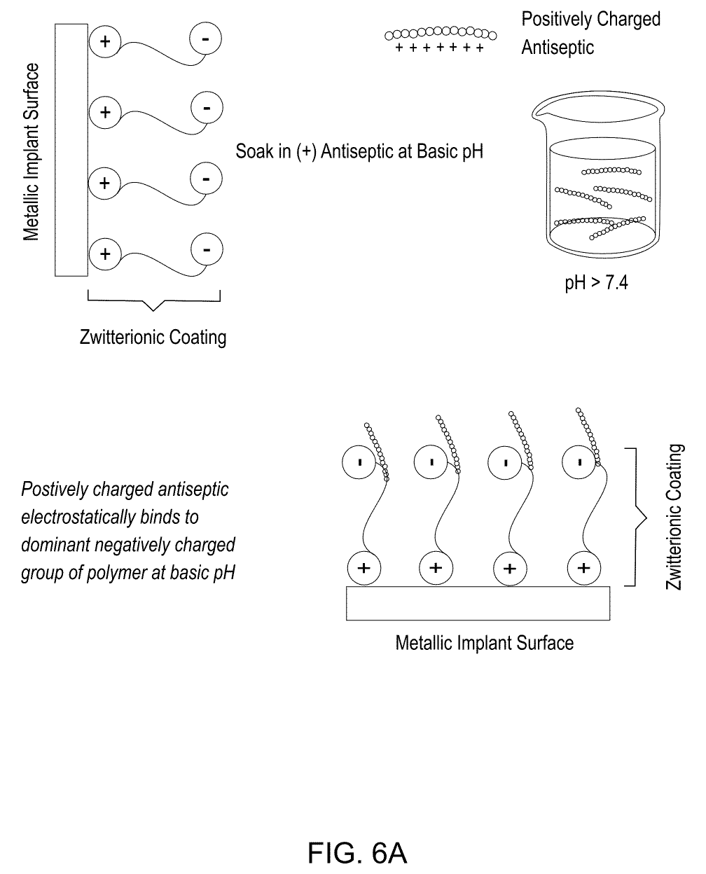

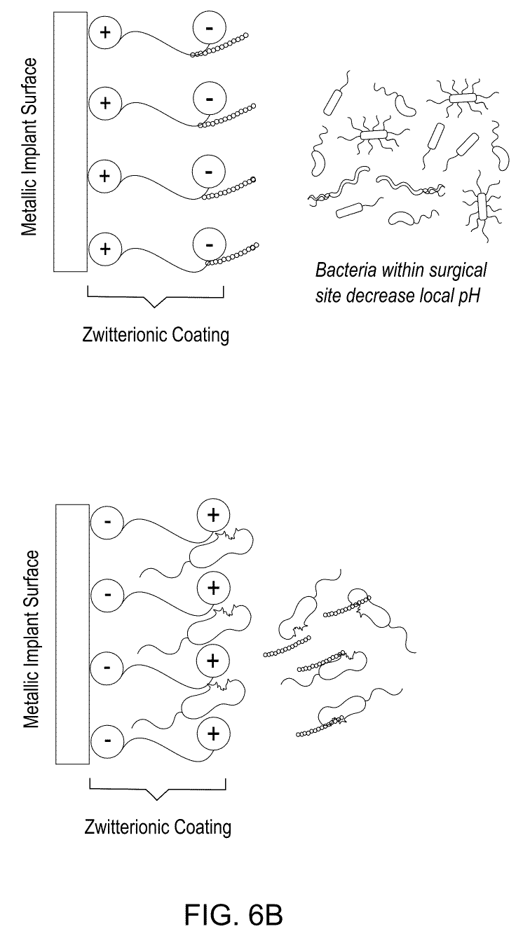

As illustrated in FIG. 6A, the zwitterionic polymer coating may be loaded with a positively charged antiseptic under non-infected conditions by soaking the implant in an antiseptic solution at a basic pH. At this pH, the negatively charged carboxylate groups dominate the polymer surface, enabling electrostatic association of positively charged antiseptic agents.

Representative antiseptics include, but are not limited to, chlorhexidine, polyhexamethylene biguanide (PHMB), and other cationic antimicrobial compounds. Electrostatic association between the antiseptic and the negatively charged polymer may enable loading of the antiseptic onto the implant prior to implantation.

### C. Infection-Triggered Release and Contact Killing

Following implantation, the coated implant may remain in a non-infected physiological environment in which the antiseptic remains bound to the zwitterionic coating. As illustrated in FIG. 6B, when bacterial colonization occurs at the surgical site, localized infection may result in a decrease in pH.

At the reduced pH associated with infection, the zwitterionic polymer undergoes a charge state transition in which the quaternary ammonium groups become dominant. This transition may lead to electrostatic repulsion of the positively charged antiseptic, thereby increasing availability or release of the antiseptic into the surrounding tissue.

Simultaneously, the exposed cationic quaternary ammonium groups on the polymer coating may provide contact-killing antimicrobial activity at the implant surface. This dual mechanism may enable rapid, localized antiseptic delivery in response to infection while supporting long-term antimicrobial protection of the implant.

### D. Dual-Function and Multi-Therapeutic Embodiments

In further embodiments, zwitterionic polymers or combinations of cationic and anionic moieties may be covalently bound to the organic acid layer to create implant surfaces capable of simultaneously binding multiple therapeutic agents having differing charge characteristics.

For example, an anionic moiety may be used to electrostatically bind or associate with a positively charged therapeutic agent, while a cationic moiety provides antimicrobial contact killing or binds a negatively charged therapeutic agent. As used herein, “contact killing” refers to antimicrobial activity that occurs upon direct contact between a microorganism and a cationic surface, including mechanisms such as disruption of microbial cell membranes, interference with membrane integrity or function, and/or inhibition of bacterial adhesion, without requiring release of an antimicrobial agent into the surrounding environment. Hydrophobic regions of the polymer coating may additionally bind nonpolar drugs or lipid-based nano- or microparticles.

In certain embodiments, proteins, glycoproteins, or antibodies may be covalently tethered to the implant surface through activation of surface functional groups, such as via N-hydroxysuccinimide (NHS) chemistry, thereby facilitating or enabling targeted therapeutic or anti-cancer applications.

### Methods of Tethering Therapeutic Agents

The therapeutic agents may be applied or associated with the functionalized implant surface using various methods, including, but not limited to, dip coating, spray coating, electrical deposition, flow coating, curtain coating, soaking, and syringe misting.

### I. Immersion-Based Application Techniques

A. Dip Coating

In dip coating, the implant is submerged into a target solution containing the therapeutic agent. The implant may be, for example, submerged at a rate ranging from approximately 1 mm/min to 200 mm/min. The implant is then gradually withdrawn from the solution at a controlled speed, which may range from approximately 0.2 mm/min to 200 mm/min. The solution may be modified to adjust viscosity and surface tension. Thickening agents such as polysaccharides, including pectin or hyaluronic acid, may be utilized to thicken the solution during withdrawal. Controlled air flow, either laminar or turbulent, may be used to promote drying of the part and influence surface loading of the therapeutic agent.

B. Soaking-Based Clinical Loading Kits

In certain embodiments, the functionalized implant is provided as part of a clinical loading kit configured for soaking-based application of a therapeutic agent. The kit may include a therapeutic agent solution container, a soaking vessel sized to receive the implant, and the functionalized implant itself.

In certain embodiments, the soaking-based clinical loading kit represents a specific implementation of the clinical loading kits described further below. Prior to implantation, the implant may be immersed in the therapeutic agent solution for a predetermined or selected period of time to allow electrostatic and/or hydrophobic association of the therapeutic agent with the polymeric coating. Following soaking, the implant may be removed from the solution and implanted at the surgical site.

In some embodiments, the soaking vessel and therapeutic agent solution are sterile and intended for intraoperative use, enabling loading of the therapeutic agent immediately prior to implantation and allowing customization of the therapeutic payload based on patient-specific or procedural considerations.

### II. Spray- and Flow-Based Applications

A. Spray Coating

In spray coating, the solution containing the therapeutic agent is atomized into droplets for application onto the implant surface. The air environment may be controlled, and vacuum conditions may be considered, if desired, to prevent contamination.

B. Syringe Misting Application

The therapeutic agent may be provided in a syringe with a spray tip and flow regulator of compressed air. The implant is sprayed with the therapeutic agent solution through the spray tip.

C. Flow Coating

In flow coating, the implants are subjected to a stream of therapeutic agent solution from an apparatus, such as tubing, that is allowed to fully flow around the components. Alternatively, several streams of therapeutic agent solution may flow onto the components. In both versions, excess solution is allowed to run off to a separate collection container.

D. Curtain Coating

In curtain coating, the implants are passed through a continuous curtain of therapeutic agent solution.

### III. Electrostatic or Field-Assisted Deposition

A. Electrical Deposition

In electrical deposition, the surface charge of the functionalized implant is enhanced by application of an external electrical field, and the implant is submerged in a target solution containing the therapeutic agent. The applied electrical field promotes electrostatic attraction between the implant surface and the therapeutic agent, thereby potentially increasing loading efficiency, uniformity, or rate of association.

Alternatively, the surface charge of the implant may be enhanced under vacuum or controlled atmospheric conditions, and the therapeutic agent solution may be sprayed or misted onto the implant while the electrical field is applied. In certain embodiments, the magnitude, polarity, waveform, and duration of the applied electrical field may be selected based on the charge characteristics of the implant surface and the therapeutic agent.

### IV. Therapeutic Agent Formulations and Clinical Loading Kits

A. Therapeutic Agent Gels

The therapeutic agent may be provided in a suspension or gel form with thickening additives such as polyethylene glycol. The implant may be soaked in the suspension or gel, or the suspension or gel may be applied via syringe misting as described above.

B. Clinical Loading Kit

In certain embodiments, the functionalized implant and therapeutic agent are provided together as a clinical loading kit configured for preoperative or intraoperative preparation of the implant. The kit may include a metallic implant having a functionalized polymeric coating as described herein and one or more containers holding a therapeutic agent solution, suspension, or gel.

The kit may further include one or more application tools selected from a soaking vessel, syringe applicator, spray device, or combinations thereof, to facilitate loading of the therapeutic agent onto the implant surface prior to implantation. In some embodiments, the kit additionally includes buffering agents or pH-modifying solutions configured to facilitate electrostatic or hydrophobic association of the therapeutic agent with the polymeric coating.

Provision of the implant as a clinical loading kit may enable therapeutic loading immediately prior to implantation, allowing customization of the therapeutic payload based on patient-specific or procedural considerations while avoiding long-term storage of pre-loaded implants. In certain embodiments, the kit is sterile and intended for single-use in a surgical setting.

While various embodiments of the present disclosure have been described in detail, it is apparent that modifications, variations, and alterations of those embodiments will occur to those skilled in the art. However, it is to be expressly understood that such modifications and alterations are within the scope and spirit of the present disclosure, as set forth in the following claims.

What is claimed is:

1. A metallic implant comprising:
a metallic substrate;
an organic acid layer covalently bound to the metallic substrate;
a polymeric coating covalently bound to the organic acid layer, the polymeric coating comprising at least one of a cationic, anionic, zwitterionic, or hydrophobic functional group; and
a therapeutic agent electrostatically or hydrophobically associated with the polymeric coating,
wherein the polymeric coating is configured to control release or availability of the therapeutic agent as a function of surface charge, hydrophobicity, or a combination thereof.

2. The metallic implant of claim 1, wherein the organic acid layer comprises a phosphonic acid.

3. The metallic implant of claim 1, wherein the polymeric coating comprises quaternary ammonium functional groups that impart contact-killing antibacterial activity.

4. The metallic implant of claim 1, wherein the polymeric coating comprises methacrylic acid functional groups that impart a net negative surface charge.

5. The metallic implant of claim 1, wherein the polymeric coating comprises a zwitterionic polymer having both cationic and anionic functional groups.

6. The metallic implant of claim 5, wherein the zwitterionic polymer comprises carboxybetaine methacrylate and exhibits a pH-dependent change in dominant surface charge.

7. The metallic implant of claim 1, wherein the therapeutic agent comprises an antimicrobial agent, a gene therapy agent, or a growth factor.

8. The metallic implant of claim 7, wherein the therapeutic agent comprises povidone iodine electrostatically associated with a cationic polymeric coating.

9. The metallic implant of claim 7, wherein the therapeutic agent comprises nucleic acid material electrostatically associated with a cationic polymeric coating.

10. The metallic implant of claim 7, wherein the therapeutic agent comprises bone morphogenetic protein-2 electrostatically associated with an anionic polymeric coating.

11. A method of forming a functionalized metallic implant, the method comprising:
providing a metallic substrate;
covalently binding an organic acid layer to the metallic substrate;
polymerizing a polymeric coating from the organic acid layer, the polymeric coating comprising at least one of a cationic, anionic, zwitterionic, or hydrophobic polymer; and
associating a therapeutic agent with the polymeric coating via electrostatic interaction, hydrophobic interaction, or a combination thereof.

12. The method of claim 11, wherein the organic acid layer comprises a self-assembled monolayer having a hydrocarbon chain length from C2 to C30.

13. The method of claim 11, wherein the polymeric coating is formed using Atom Transfer Radical Polymerization (ATRP) or Reversible Addition–Fragmentation Chain Transfer (RAFT) polymerization.

14. The method of claim 11, further comprising grafting a polymerization initiator to the organic acid layer prior to polymerization.

15. The method of claim 11, wherein the therapeutic agent is applied to the polymeric coating by soaking, spraying, dip coating, or electrical deposition.

16. A method of using a functionalized metallic implant, the method comprising:
implanting a metallic implant at a surgical site, the metallic implant comprising a metallic substrate having a covalently bound polymeric coating that includes at least one of cationic, anionic, zwitterionic, or hydrophobic functional groups;
contacting bacteria present at, or migrating to, the surgical site with the polymeric coating, such that bacterial cell membranes are disrupted or bacterial adhesion to the implant is inhibited; and
releasing or making available a therapeutic agent associated with the polymeric coating into surrounding tissue in response to one or more local biological conditions.

17. The method of claim 16, wherein release of the therapeutic agent is triggered by a change in local pH associated with infection.

18. The method of claim 16, wherein the polymeric coating is zwitterionic and releases a positively charged antiseptic in response to acidic conditions.

19. The method of claim 16, wherein the metallic implant is an orthopedic, dental, spinal, trauma, or tumor implant.

20. The method of claim 16, wherein the polymeric coating remains permanently bound to the metallic substrate and does not elute from the implant.
